(19) United States
(12) Patent Application Publication
(10) Pub. No.: US 2024/0207219 A1
(43) Pub. Date: Jun. 27, 2024

(54) Artemisia anti-bacterial compositions and methods of use for inhibition of cells of Mycobacterium species

(71) Applicants: Worcester Polytechnic Institute, Worcester, MA (US); The Penn State Research Foundation, University Park, PA (US)

(72) Inventors: Scarlet S. Shell, Shrewsbury, MA (US); Pamela Weathers, Stow, MA (US); Joshua J. Kellogg, University Park, PA (US)

(21) Appl. No.: 18/392,419

(22) Filed: Dec. 21, 2023

Related U.S. Application Data
(60) Provisional application No. 63/543,591, filed on Oct. 11, 2023.
(60) Provisional application No. 63/434,500, filed on Dec. 22, 2022.

(51) Int. Cl. A61K 31/352; A61K 36/282; A61K 45/06; A61K 49/00; A61P 31/06

(52) U.S. Cl. CPC A61K 31/352; A61K 36/282; A61K 45/06; A61K 49/0008; A61P 31/06; A61K 2236/15; A61K 2236/39

(57) ABSTRACT

Artemisia annua and A. afra dichloromethane extracts contain bactericidal activity against Mycobacterium tuberculosis (Mtb) strain mc²6230 under natural infection stress conditions: carbon source metabolism (glycerol, dextrose, and cholesterol) and hypoxia. Significant bactericidal activity against Mtb was observed regardless of carbon source. Extracts from A. afra showed the highest bactericidal activity against Mtb for tested carbon sources, and A. annua bactericidal activity was greatest against Mtb in minimal media with glycerol. A. annua and A. afra. extracts were bactericidal against Mtb under hypoxic conditions. Growth was halted and viability diminished several logs-fold under hypoxic conditions in the presence of each extract. Transcriptomic analysis revealed that A. afra exerts different effects on Mtb than artemisinin, indicative of phytochemicals in A. afra with unique modes of action. Biochemometric analysis of A. afra resulted in isolation of a methoxylated flavone (compound 1), with considerable activity against Mtb strain mc²6230.

### RELATED APPLICATIONS

The present application claims the benefit of U.S. provisional application No. 63/434,500, filed Dec. 22, 2022, entitled “TUBERCULOSIS TREATMENT” and U.S. provisional application No. 63/543,591, filed Oct. 11, 2023, entitled “A METHOXYLATED FLAVONE FROM ARTEMISIA AFRA KILLS MYCOBATERIUM TUBERCULOSIS”, each of which is hereby incorporated herein by reference in its entirety.

### STATEMENT OF FEDERALLY SPONSORED RESEARCH AND DEVELOPMENT

This invention was made, at least in part, with government support under Grant No. AI151481 awarded by the National Institutes of Health and under Hatch Act Project No. PEN04772 awarded by the United States Department of Agriculture/NIFA. The Government has certain rights in the invention.

### FIELD OF THE INVENTION

Compounds extracted from plants of genus Artemisia are provided that have activities against tuberculosis bacilli and related pathogens.

### BACKGROUND

Tuberculosis (TB), one of the major fatal diseases of humanity, is a major health, social, and economic burden worldwide and occurs mainly in low and middle-income countries. TB remains a major health challenge even after its first documented case 3,000 years ago (Barberis et al., 2017, J. Preventinve Med and Hyg 58(1), E9) and 130 years after the discovery of Mycobacterium tuberculosis (Mtb), the causative agent of TB. According to the WHO (WHO, 2022) one of the top 10 global causes of death is tuberculosis (TB) with 10.6 million reported cases and 1.6 million deaths in 2021.

Drug-resistant TB requires significantly longer treatment regimens with additionally incapacitating reactions and diminishing results with only a few drugs (linezolid, bedaquiline, delamanid, and pretomanid) available as antitubercular agents to treat multidrug-resistant TB (Keam, 2019, Drugs 79(16), 1797-1803; Mokrousov et al., 2019, Clin. Microbiol. Infect. 25(10), 1295-1297; Polsfuss et al., 2019, Clin. Infect. Dis. 69(7), 1229-1231; The main cause of persistent Mtb infection is formation of hypoxic granulomas inside the host where it can survive for a longer period in a dormant stage further complicating treatment (Singh et al., 2020, J. Bacteriol. 202(7) e00705-00719). The emergence of resistance to these drugs demands the discovery of new drugs and combinations to expand TB therapy at all stages of the disease.

The development of novel therapies is linked with an understanding of different survival strategies by bacterial pathogens to overcome stress. Bacterial nutrition is one of the main aspects of determining the host-pathogen interaction (Ehrt et al., 2018, Nature Rev. Microbiol. 16(8), 496-507) and thus is useful in drug development. The infection pattern of bacterial pathogens and the interaction between drugs and Mtb nutritional requirements may greatly affect disease progression. Mtb uses lipids of the host stored in functional lipid bodies during infection and intracellular replication suggesting the importance of lipids during infection (Lovewell, R. R et al. Curr. Opin. Microbiol. 29, 30-36).

Although lipids are Mtb's major carbon source in-vivo, their low solubility and the presence of multiple carbon sources led to the consideration that there might be some other carbon sources bacteria use during infection (Serafini et al., 2019, Mol. Microbiol. 112(4), 1284-1307). Different carbon sources are co-catabolized by Mtb in-vitro (Koh, E.-I. et al., 2022, Proc. Natl. Acad. Sci. 119(15), e2201632119). Kalia et al. (Kalia, N. P., et al., 2019 Sci. Rep. 9(1), 1-9) confirmed that during infection, mycobacteria use multiple carbon sources.

Plant extracts have been used for treating diseases for millennia and about 100,000 plant species have medicinal value, often without adverse side effects on human health and the environment (Behl, T. et al., 2021, Pharmaceuticals 14(4), 381) consistent with human evolutionary history of use of plants for food. Plant specialty molecules (previously termed plant secondary metabolites; PSM) can affect the microbial cell in several different ways including disruption of membrane function and structure, interruption of DNA/RNA synthesis and function, interference with intermediary metabolism, induction of coagulation of cytoplasmic constituents, and interruption of normal cell communication. Antibiotics currently used as therapies to treat pathogenic diseases rely on the mechanism of bacterial growth inhibition. These factors are important for development of novel therapies from plants to fully understand mechanisms of antimicrobial activity of plant compounds.

African wormwood (Artemisia afra) is a member of a prolific biosynthetic genus; the Dictionary of Natural Products contains ca. 1500 records for the genus Artemisia. This genus of more than 500 species is a source of important bioactive compounds, the most famous of which is artemisinin, the noted antimalarial produced by Artemisia annua L (Maciuk, A., et al., 2023, Nat. Prod. Rep. 40(6), 1130-1144; Trifan, A. et al., 2022, Antioxidants 11(5) 1017; Weathers, P. J. 2022, Nat. Prod. Reports 40: 1158-1169 and incorporated herein by reference in its entirety). While both Artemisia species A. annua and A. afra contain a plethora of specialty molecules, A. afra typically contains no detectable artemisinin (Liu, N. Q. et al., 2009, S. African J. Botany 75(2), 185-195). Also similar to A. annua, A. afra has a long history of ethnobotanical medicinal use, primarily in southern Africa rather than South Asia (Ibid.).

Many species of Artemisia (FIGS. 1A, 1B) have different proven medicinal properties and are used for treating diseases like malaria, cancer, and hepatitis (Abad, M. J. et al., 2012, Molecules 17(3), 2542-2566; Ekiert, H. et al., 2022, Molecules 27(19) 6427; Trendafilova, A. et al., 2020 Foods 10(1), 65), and have even shown efficacy against covid (Nair, M. S. et al., 2021, J. Ethnopharm. 274, 114016; ; Nair et al., 2022, J. Ethnopharm.284, 114797; Nair et al., 2023 J. Ethnopharm. 308, 11691). Traditional treatments led to testing of different Artemisia species and their extracts against several pathogens including mycobacteria in vitro, and in vivo (Cantrell, C. et al., 1998 Phytomed. 5(2), 137-145; Uba, A. et al., 2003, African J. Pharm. Sci 6(1), 15-19). A. annua and A. afra are used globally to treat fever and cou vo in a murine model of tuberculosis gh which are the common symptoms of many diseases including TB (Thring, T. et al., 2006 J. Ethnoharmacol 103(2) 261-275). A. annua produces the antimalarial drug, artemisinin (ART), which also has antitubercular activity (Martini, M. et al., 2020, J. Ethnopharmacol. 262, 113191). ART targets the main survival strategy for Mtb during non-replicating perseverance of Mtb persistence by blocking the two-component regulatory system i.e. DosRST under hypoxic conditions in-vitro (Zheng, H. et al., 2019 ACS Chem Biol. 15(1), 52-62). Although it produces little to no ART, A. afra also significantly inhibited Mtb growth (Mativandlela, S. et al., 2008 Phytother. Res. 22(6), 841-845; Ntutela, S. et al., 2009 Tuberculosis 89, S33-S40).

A. annua and A. afra dichloromethane (DCM) extracts were found to have strong bactericidal activity against M. tuberculosis (Martini et al., 2020, Ibid.). There is a need for additional information regarding whether these plants contain bactericidal compounds beyond ART. Further, there are no reports regarding the efficacy of A. annua and A. afra against Mtb in the presence of different carbon sources and under hypoxia.

A greater emphasis on long-term treatments of tuberculosis is direly needed. Currently, susceptible tuberculosis infections require a regimen consisting of an intensive phase of two months of isoniazid, rifampin, pyrazinamide, and ethambutol followed by a second sustained phase of four months of isoniazid and rifampin combination therapy (Chauhan, A. et al., 2021 Life Sci. 274 119301). Long periods of multi-drug exposure are needed to slow the emergence of resistant Mtb and kill persistent, non-replicating bacteria, which have greatly reduced drug sensitivity but will cause relapse if not fully cleared. As multidrug resistant (MDR), extensively drug resistant (XDR), and total drug resistant (TDR) strains emerge, current first-line treatments are not sufficient, requiring longer treatment regimens^{3,4} Even in cases of fully drug sensitive TB, the length and complexity of the first-line regimen creates barriers in the resource-limited settings where the disease is most prevalent.

There is an urgent need to meet the evolving challenge of tuberculosis today and meet the WHO's goals, by developing new drugs.

### SUMMARY

An aspect of the invention herein provides a method of use of 5-hydroxy-7-methoxy-2-(4-methoxyphenyl)chromen-4-one (e.g., 5-hydroxy-4′,7-dimethoxyflavone), identified herein as composition 1, for inhibiting growth and viability of bacteria, the method comprising contacting the bacteria with composition 1 and observing growth and viability in comparison to control bacteria not contacted. For example, the bacteria are cells of genus Mycobacterium, particularly the cells are M. tuberculosis, Mtb. Species previously identified as genus Mycobacterium are recently classified taxonomically as Mycobacterioides, according this alternative genus name is included herein. Other mycobacterial species previously classified in genus Mycobacterium are now classified as Mycolicibacterium, e.g., M. fortuitum. Accordingly these taxonomic genus designations which have changed historically may be considered equivalent for any particular previously named mycobacterial species.

In alternative embodiments, the bacterial cells of genus Mycobacterium are classified as other pathogenic mycobacterial species within members of this genus, i.e., the cells are from at least one selected from the group of species: M. abscessus, M. avium-intraceliulare, M, chelonae, M. fortuitum, M. kansasii, M. xenopi, M. marnum, M. scrofulaceum, and M. szugai.

An embodiment of the method of contacting the bacteria includes treating an infection in a subject in need of treatment of a pathogenic fungus. The subject can be an animal, for example a mammalian subject, for example a human subject, or high value bird, or even a plant. In a preferred embodiment the subject is a human patient. Alternatively, the subject is a mammalian species in a clinical trial, for example, rodent, guinea pig, veterinary patient such as a dog or cat or horse, high value zoo animal. In another embodiment the subject is an invertebrate that serves as a model of a fungal disease, such as brine shrimp, fruit fly, round worm.

For treating the infection, an embodiment of the method further includes, prior to the step of contacting, formulating the composition 1 with at least one additional component selected from the group of: a second antibacterial agent, a pharmaceutically acceptable buffer, an emollient, and a carrier. For treating the infection, an embodiment of the method further includes formulating the composition 1 in an effective dose. Administering the effective dose to the mammalian subject in various embodiments is delivering composition 1 by a route selected from the group of: intranasal, oral, sublingual, intravenous, intradermal, transdermal, topical, intra-muscular, and subcutaneous.

The inventors have analyzed Mtb sensitivity to composition 1 and have determined that the anti-mycobacterial activity for cells grown in limiting oxygen is enhanced compared to aerobically grown cells. The significance is that in a disease such as tuberculosis the cells have more limited access to oxygen in vivo during the infection. Accordingly, in various embodiments, the pharmaceutical composition 1 has a minimal inhibitory concentration (MIC) for M. tuberculosis of less than 600 micrograms/ml; 300 micrograms/ml; less than 100 micrograms/ml; less than 50 micrograms/ml.

An aspect of the invention provides a method of making composition 1 from leaves of an Artemisia plant species. Composition 1 chemically is 5-hydroxy-7-methoxy-2-(4-methoxyphenyl)chromen-4-one (e.g., 5-hydroxy-4′,7-dimethoxyflavone), the method comprising: providing a sample of leaves of A. afra or A. annua; grinding under sonication of the leaves under dichoromethane (DCM), thereby extracting the composition 1 into the supernatant; fractionating the supernatant by flash chromatography and eluting with a gradient of solvents; and, sub-fractionating the activity containing fraction by reverse phase flash chromatography, to obtain the composition 1.

In various embodiments of this method, the leaves provided are from plants of A. afra. For example, the sample is at least about 5 mg, about 5 grams, about 50 grams, about 500 grams, about 1 kg, about 2 kg. In further embodiments, eluting with a gradient of solvents is using hexane, chloroform and methanol in a series of ratios. In further embodiments, the reverse phase flash chromatography includes eluting with acetonitrile, water and chloroform in a series of ratios. The method further includes determining the chemical structure of the resulting composition 1 to be a 5-hydroxy-4′,7-dimethoxyflavone. The method yields a yellowish powder. The method further includes steps of analyzing purity and identifying the chemical structure. The steps of identifying chemical structure determined that composition 1 is 5-hydroxy-7-methoxy-2-(4-methoxyphenyl)chromen-4-one. Determining the purity of the yellowish powder shows that this material is at least 90%, at least 95%, or at least 97% pure. The method further includes in various embodiments diluting the sub-fraction having activity or dissolving the yellowish powder and diluting the solution, for performing toxicity studies. The steps of performing toxicity studies includes at least one selected from the group of contacting: cells of mammalian origin in vitro, viz., in cell culture in Petri plates; or contacting mammalian subjects in vivo; and contacting non-mammalian subjects in vivo.

### BRIEF DESCRIPTION OF THE DRAWINGS

FIGS. 1A and 1B are photographs of leaves of each of the Artemisia plants, FIG. 1A is A. annua and FIG. 1B is A. afra, used in the methods herein.

FIGS. 2A and 2B are survival curves as a function of time of Mtb cells grown with one of several carbon sources, with and without extracts of each of the Artemisia species. Comparative growth of Mtb in the presence of 5 mg/ml A. afra extracts in each of the carbon sources is shown in the FIG. 2A. Comparative growth of Mtb in the presence 75 μg/ml A. annua extracts in all carbon sources is shown in FIG. 2B. Controls of cells grown absent drug show increases in cell numbers as a function of time, the four upper data plots in each figure. Following are the single carbon sources used in this experiment: 7H9+glycerol, minimal medium (MM)+glycerol, MM+dextrose, and MM+cholesterol. Data are the average from three independent experiments.

FIG. 3A is a plot as a function of time showing comparative analysis of Mtb growth in A. afra and A. annua under hypoxic conditions. In the hypoxia experiment, a limited amount of oxygen was provided to each of three cultures of Mtb in the presence same volume of either control DMSO, A. annua and A. afra extracts. (N=3; bars=±SE; *=P<0.05; ** =P<0.01; *=P<0.001; ****=P<0.0001).

FIG. 3B shows survival as a function of time of Mtb treated with an A. afra extract and constituent fractions and subfractions in anerobic conditions. Treatment was applied to liquid cultures at Day 0 and aliquots were plated on drug-free solid media at the indicated timepoints. Colonies were enumerated after 3-4 weeks of growth in order to quantify the number of colony forming units (CFU) that had survived drug treatment at each timepoint. Control no-drug samples were given DMSO as a vehicle control. Mtb was exposed to crude DCM extract of A. afra and the three flash chromatography fractions with the lowest MICs.

FIG. 3C shows results of Mtb exposed to crude DCM extract of A. afra and subfraction HAB9. No viable colonies were recovered from cultures treated with HAB9 after six days. The limit of detection is indicated with a dashed line.

FIG. 3D shows data from hypoxic Mtb sealed in vials with 17 mL of culture and 11 mL of headspace. The cultures became hypoxic approximately eight days after sealing as evidenced by methylene blue decoloration. After the cultures had been sealed for 14 days, subfraction HAB9 or the vehicle control DMSO were injected by needle to prevent introduction of oxygen. This was considered day 0 of treatment. No colonies were recovered from cultures treated with HAB9 after two or six days of treatment. The limit of detection is indicated with a dashed line.

FIG. 4A displays the results of Principal Component Analysis (PCA) used to determine whether A. afra, A. annua, and artemisinin have distinct transcriptomic impacts on Mtb. Aerobically growing Mtb was treated with each plant extract or the pure compound at lethal doses for four hours or at inhibitory and sub-inhibitory doses for 24 hours. Untreated control cultures were harvested at the same two time-points and RNAseq was used to generate transcriptomic profiles. All conditions were tested in quadruplicate. PCA was done on the read count tables from each sample in each condition, revealing that each treatment clustered separately.

FIG. 4B displays expression of each gene in each treatment compared to that in the time-matched control, and differentially expressed genes were subject to Gene Set Enrichment Analysis. Gene sets were GO Biological Process gene lists obtained from AmiGO.¹⁵ “Activated” gene sets had higher expression in the treated samples compared to the controls while “suppressed” genes sets had lower expression. “GeneRatio” indicates the proportion of genes in the set that were differentially expressed in the indicated condition. “p.adjust” is the P value of the overrepresentation of genes within the set among the differentially expressed genes, after correction for multiple comparisons.

FIG. 5 is an Upset plot summarizing the commonalities in gene expression changes across treatments. Genes were classified as differentially expressed if their log 2 fold change in mRNA abundance in a treatment compared to its control was >1 or <−1 with an adjusted p value<0.05. The total number of differentially expressed genes in each treatment is shown on the lower left (“set size”). “Inclusive intersection size” (main histogram) indicates the number of genes that were differentially expressed in any two or three treatments. “Inclusive” means that genes were included in a set even if they also were present in other sets.

FIG. 6 shows heteronuclear single quantum correlation (HSQC), and heteronuclear multiple bond correlation (HMBC) NMR data permitted the identification of compound 1 as defined here, as 5-hydroxy-7-methoxy-2-(4-methoxyphenyl)chromen-4-one (e.g., 5-hydroxy-4′,7-dimethoxyflavone).

### DETAILED DESCRIPTION OF THE EMBODIMENTS

In the examples herein, efficacy of extracts of A. annua and A. afra for antibacterial activities was measured with Mtb grown in different carbon sources and under hypoxic conditions to evaluate the potential of these plant extracts for the treatment of tuberculosis.

Data were obtained in Examples herein using Mtb, however methods and uses are applicable to other strains of Mycobacterium and species of other mycobacterial genera, including for example, M. abscessus, M. avium-intracellulare, M. chelonae, M. fortuitum, M kansasii, M xenopi, M. rarinur, M. scrofulaceum, and M. szulqai.

All solvents and chemicals used in Examples herein were of reagent or spectroscopic grade, as required, and obtained from VWR (Radnor, PA, USA) or Sigma Aldrich (St. Louis, MO, USA).

### Plant Materials

Dried leaves of Arenisia annua L. cv. SAM (MASS 317314) were harvested from plants field-grown in Stow, MA from rooted cuttings. Alternatively for extraction several kg of dried leaves of Artemisia afra Jacq. ex Willd. cv. MAL (voucher FTG181107; B #1RbA.10.12.20) were obtained from Atelier Temenos LLC (Homestead, FL, USA). For transcriptomic, Mtb, and toxicity analyses dried leaves of A. annua L. SAM cultivar (voucher MASS 00317314) and the A. afra MAL cv. were used. Leaves were harvested and processed as described by Weathers and Towler (Weathers, P. J. et al., 2014, id Crops Prod. 62, 173-178). Dried leaves of Artemisia afra Jacq. ex Willd. cv. NIAL (originated from Malawi; FTGi 81107; B #1RbA.10 12.20) was obtained from Atelier Temenos LLC in Homestead, FL. The vouchers for other cultivars of A. afra are SEN (LG0019529), LUX (MNHNL17730), and PAR (LG0019528). DCM extracts were pooled and dried under N₂ as previously detailed (Desrosiers, M. R. et al., 2020 Biomolecules 10(2) 254) and ART was analyzed using GC-MS as described in Kane et al. (Kane. N. F. et al.. 2022 J. Ethnopharmacol. 115587). Hot water tea infusions were also prepared as detailed in Desrosiers et al. (Desrosiers et al., Ibid.). ART content of SAM was 17.08 mg/g dry leaf mass but was undetectable in MAL. Because A. afra MAL has no detectable ART and is now readily available in the US, it was used in subsequent experiments instead of the other cultivars unless otherwise indicated and especially the SEN cv that has a small amount of ART as reported in Martini et al. (Martini et al., 2020). Hot water and DCM extracts were prepared according to Kane et al. (Ibid.) and Martini et al., (Ibid.) respectively. For transcriptomic, Mtb, and toxicity analyses dried leaves of A. annua L. SAM cultivar (voucher MASS 00317314) and the A. afra MAL cv. were used.

Bacterial strains and culture conditions Mycobacterium tuberculosis (Mtb) cells used were auxotrophic mc²6230 (ApanCD, ΔRD1 (Sambandamurthy, V. K. et al., 2006 Vaccine 24 (37) 6309-6320), like mc²6030 but lacking hygromycin resistance¹⁸, and were grown at 37° C. and 200 rpm in Middlebrooks 7H9 broth supplemented with 10% OADC (0.5 g/L oleic acid, 50 g/L bovine serum albumin fraction V, 20 g/L dextrose, 8.5 g/L sodium chloride, and 40 mg/L catalase), 0.2% glycerol, 0.05% Tween 80 and 24 ug/mL pantothenate. Where specified, strains were grown in minimal media (0.5 g/liter asparagine, 1 g/liter KH₂PO₄, 2.5 g/liter Na₂HPO₄, 50 mg/liter ferric ammonium citrate, 0.5 g/liter MgSO4.7H₂0, 0.5 mg/liter CaCl₂, and 0.1 mg/liter ZnSO4) supplemented with 0.1% tyloxapol, 24 ug/mL pantothenate, and 0.1% glycerol or 0.1% dextrose or 0.1% cholesterol. Middlebrooks 7H10 supplemented with 0.5% glycerol, OADC and 24 ug/mL pantothenate was used to grow Mtb on solid media. Once this medium was established as suitable in Examples herein, liquid cultures were grown in 50 mL conical tubes in Middlebrook 7H9 broth, supplemented with 10% OADC (final concentrations 0.05 g/L oleic acid, 5 g/L bovine serum albumin fraction V, 2 g/L glucose, 0.85 g/L sodium chloride, and 4 mg/L catalase), 0.2% glycerol, 0.05% Tween 80, and 24 μg/mL pantothenate (7H9) at 37° C. and 200 rpm, in ambient light. To determine cell numbers of viable surviving cells, colony forming units (CFUs) were determined by plating aliquots of the cultures on Middlebrook 7H10 solid media supplemented with 0.5% glycerol, OADC, and 24 μg/mL pantothenate (7H10).

### Determination of the Minimum Inhibitory Concentration (MIC)

MICs of Mtb strain mc²6230 for DCM extracts of A. annua and A. afra SEN cv using a resazurin microtiter assay (REMA) were previously reported (Martini et al., Ibid.). Using the same method, the MIC for MAL was measured and remeasured SEN as well as for the water extracts (tea infusions) of A. annua cv. SAM, and A. afra cv. PAR, LUX and SEN MICs for Artemisia extracts are expressed as the amount of leaf dry mass represented in the tested aliquot of DCM extract.

Determining the bactericidal activity of A. afra and its fractions against Mtb For aerobic growth analyses, Mtb was grown in 7H9 until the bacteria reached exponential phase. The OD₆₀₀ was adjusted to 0.1 and then an A. afra plant extract or a fraction thereof was added to each of triplicate cultures at the indicated concentrations. Triplicate cultures without the addition of drugs were used as a control. Samples were taken at 0, 2, 4, 6, and 10 days of incubation after addition of drugs, then serially diluted in 7H9 and plated on 7H10. Colonies were counted after 21 to 28 days of incubation at 37° C. and expressed as CFU/mL culture.

A variation of the Wayne model²⁸ was used to determine the bactericidal activity of A. afra fraction HAB9 in hypoxic growth conditions. Mtb was grown in 7H9 without oleic acid until bacteria reached exponential phase and then the OD₆₀₀ of the culture was adjusted to 0.1.17 mL of culture was added to 20 mL serum bottles (Wheaton, actual volume 28 mL). Bottles were sealed using a vial crimper with rubber caps (Wheaton, W224100-181 Stopper, 20 mm) and aluminum caps (Wheaton, 20 mm aluminum seal) and cultures were grown at 37° C. with 125 rpm shaking. Methylene blue at 1.5 μg/mL was added to separate cultures and its discoloration was used as an indicator of hypoxic conditions (usually after eight days). Triplicate cultures were opened 14 days after sealing the vials, serially diluted in 7H9, and plated on 7H10 to enumerate the initial CFU/mL. Further, at a time point of 14 days after sealing the vials, A. afra subfraction HAB9 diluted in DMSO, or an equivalent volume of DMSO as a no-drug control, was aseptically added to each of separate cultures at a final concentration 0.625 mg/mL using a syringe and a 30G needle to avoid the introduction of oxygen. After 2 and 7 days of drug treatment, vials were opened and plated to determine CFUs as described herein.

### A. afra and A. annua Bactericidal Activity Against Mtb Cells Cultured in Different Carbon Sources

Mtb was grown in 7H9media+glycerol as described previously and when they reached a required OD₆₀₀=0.8, cells were centrifuge, washed twice with PBS, and resuspended in 7H9 with glycerol or in minimal media containing 0.1% glycerol, 0.1% dextrose or 0.1% cholesterol and 0.1% tyloxapol. After 48 hrs, each log-phase culture was adjusted to a final OD₆₀₀ of 0.1. Mtb suspensions were inoculated in triplicate with a final concentration of two times the MIC values as determined for A. annua (75 μg/mL) or A. afra (5 mg/ml) extracts diluted in DMSO. Controls consisting of media with Mtb without added extracts were included also in triplicate. In all cases, the final volume in each conical tube was 5 mL. Mtb cultures were incubated shaking at 250 rpm and 37° C. Mtb samples were taken from all conditions at day 0, 2, 4, 6 and 10 of incubation and then plated on 7H10. After 15-20 days, resulting Mtb colonies were counted in each condition and the CFU/mL was calculated.

### A. afra and A. annua Bactericidal Activity Against Mtb Cultured Under Hypoxia

Mtb was grown in 7H9 broth supplemented with 10% ADC (50 g/L bovine serum albumin fraction V, 20 g/L dextrose, 8.5 g/L sodium chloride, and 40 mg/L catalase), 0.2% glycerol, 0.05% Tween 80 and 24 ug/mL pantothenate at 200 rpm at 37° C. Exponential phase cultures were diluted to OD₆₀₀=0.1. Each Mtb culture was sealed in a glass bottle with a tightly sealed cap and agitated at 120 rpm at 37° C. Methylene blue was used as an indicator of hypoxic conditions. Six days after hypoxia was established, cultures were treated with the two times the MIC values as determined for A. annua (75 μg/mL) or A. afra (5 mg/ml) extracts. The extracts were injected aseptically using a 0.3 mm syringe to minimize the introduction of oxygen. The same volume of DMSO was also injected under the same conditions to use as a control. The hypoxic Mtb cultures were incubated shaking at 120 rpm and 37° C. for 2 or 7 days and finally plated on solid media as previously described. Mtb cultures were also plated before adding the respective drugs. All conditions were tested in triplicates and CFU/mL were counted after 15-20 days.

### Results Determined for the Minimum Inhibitory Concentration (MIC)

Extracts of both hot water and DCM extracts of both Artemisia sp. showed strong bactericidal effects against Mtb. Hot water extracts ranged from 1.3-1.7 mg/mL for the A. afra cultivars SEN, PAR and LUX; A. annua was 1.9 mg/mL. A hot water extract of MAL was not measured. MICs for DCM extracts of A. afra SEN and MAL were 4.8-10 mg/mL and 2.5, respectively. DCM MICs for SEN and MAL were also similar within the range of the assay and since in contrast to SEN, MAL was ART-free and readily available as a clonal source in the US, experiments were continued using the MAL cultivar. Considering that MICs of DCM extracts were not that different from the hot water extract MICs, yet allowed extracts to be more concentrated, further experiments were conducted using the DCM extracts.

Effect of Growth of Mtb in Different Carbon Sources on A. afra and A. annua Bactericidal Activity

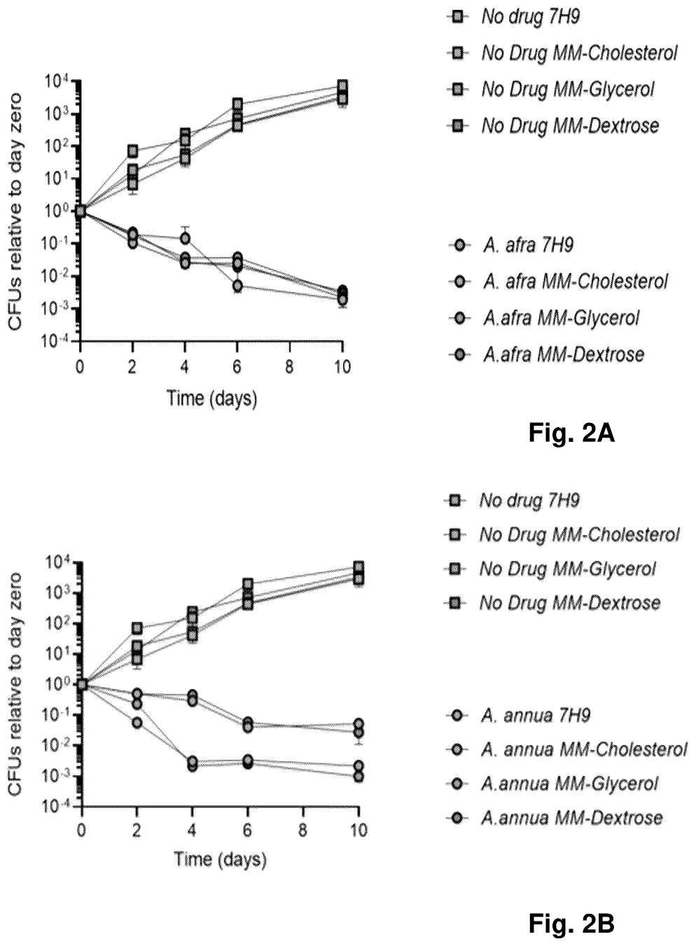

A. annua and A. afra DCM extracts were tested for their bactericidal activity against Mtb strain mc²6230 in three different carbon sources: glycerol, dextrose and cholesterol. Untreated Mtb grew at equivalent rates and to equivalent final yields in all three carbon sources (FIGS. 2A-2B). Based on the killing curves, the results revealed major growth differences of Mtb in different carbon sources when exposed to A. annua and A. afra extracts. A. afra extracts showed similar bactericidal activity in all the tested carbon sources, as there were no significant differences in its bactericidal activity when Mtb grow using glycerol, dextrose or cholesterol (FIG. 2A). However, the bactericidal activity was lower but still significant when grown in cholesterol and dextrose in comparison to glycerol as sole carbon source when cells were treated with the A. annua extract suggesting different effects of carbon metabolism on the bactericidal effect of both extracts. (FIG. 2B).

### A. afra and A. annua Bactericidal Activity Against Mtb Under Hypoxia

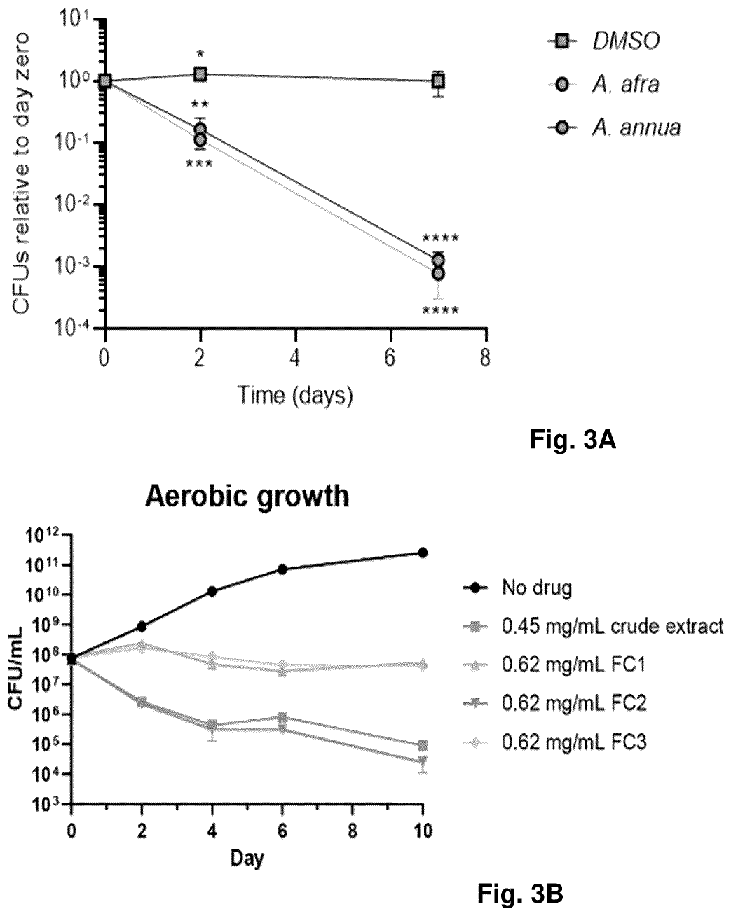

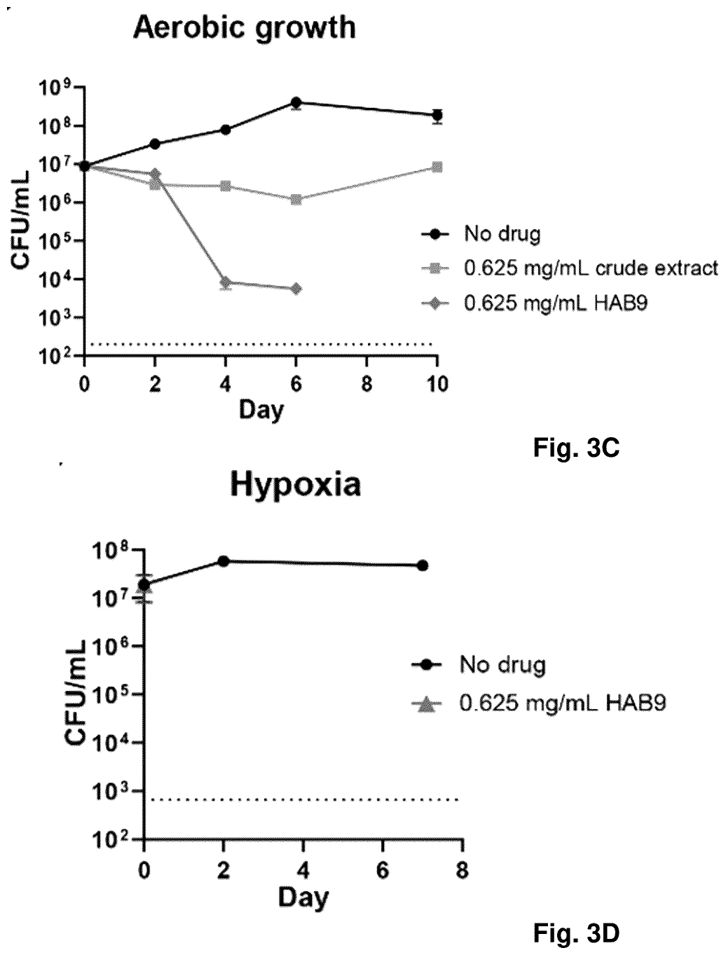

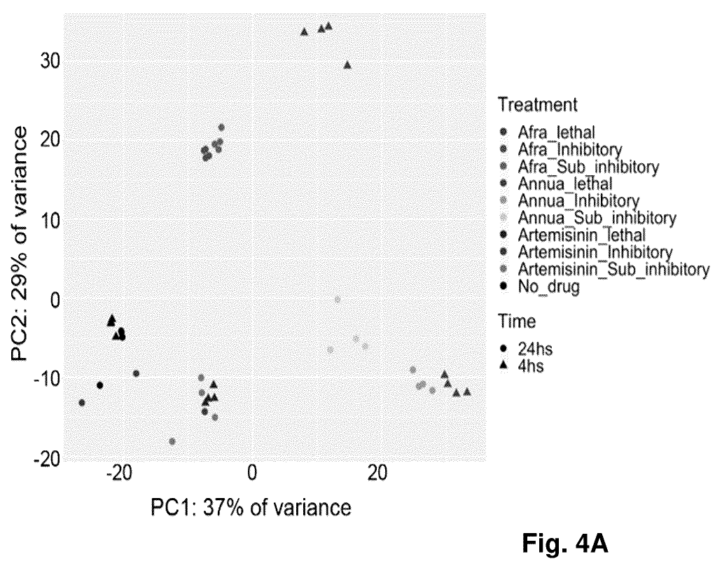

Considering that Mtb survives under hypoxic conditions within lung granulomas during natural infection, A. annua and A. afra extracts were tested for their bactericidal activity against Mtb grown under hypoxic conditions. Over 7 days, the viable population of Mtb remained relatively constant in cultures treated with DMSO only. We observed a small increase in the number of viable cells on day 2 treated only with DMSO may be due to minimal oxygen introduction during drug injection or to a small amount of remaining oxygen in the bottles. However, the viable Mtb cell population declined by one and three orders of magnitude at 2 and 7 d, respectively, after incubation in hypoxic conditions and treated with A. afra or A. annua DCM extracts (FIG. 3A). DMSO was also injected as a control because it was used a solvent to dissolve the plant extracts. According to our results, we can conclude that A. afra and A. annua have the same bactericidal effect under hypoxic conditions.

### RNAseg

Mtb cultures were grown to exponential phase, diluted to OD=0.1, and treated for four hours with lethal levels or 24 hours with inhibitory and sub-inhibitory levels of A. annua extract, A. afra extract, or artemisinin. Untreated control cultures were harvested at the same timepoints. Pure artemisinin treatments were 150 μg/mL (lethal, 2X MIC), 100 μg/mL (inhibitory, 1.33X MIC), and 37.5 μg/mL (sub-inhibitory, 0.5X MIC). The extract concentrations for these analyses were expressed in terms of the dry leaf mass used to make the extracts as done in publications (Martini, M. C. et al., 2020 Ibid.; Kiani, B. H. et al. 20203 Ibid.), rather than the mass of the extract itself as done for the other analyses herein. A. annua treatments were the extract from 9 mg dry leaf mass per mL culture (lethal, 2X MIC, and resulting in 75 μg/mL artemisinin), 4.5 mg dry leaf mass per mL culture (inhibitory, MIC, and resulting in 37.5 μg/mL artemisinin), and 2.25 mg dry leaf mass per mL (sub-inhibitory, 0.5X MIC, and resulting in 18.25 μg/mL artemisinin). A. afra treatments used for cultures were the extract from 5 mg dry leaf mass per mL culture (lethal, 2X MIC), 2.5 mg dry leaf mass per mL culture (inhibitory, MIC), and 1.25 mg dry leaf mass per mL (sub-inhibitory, 0.5X MIC). The A. afra DCM extract residue weight was approximately 9% of the dry leaf mass, and the actual extract concentrations in the corresponding cultures were therefore approximately 0.45 mg/mL, 0.225 mg/mL, and 0.113 mg/mL, respectively. Quadruplicate cultures were used for each condition. Five mL of culture was used for each 24-hr RNA extraction and 20 mL of culture was used for each 4-hr RNA extraction. RNA was extracted as described (Bar-Oz, M. et al. 2023 PLOS Pathogens 19(8) e1011575 except that 100 μm zirconium beads (OPS Diagnostics) were used for lysis; rRNA was depleted and paired-end Illumina sequencing libraries were constructed as described (Culviner, P. H., et al. 2018 Molec. Cell 70 (5) 868-880; Culviner, P. H. et al. mBio 11(2) https://doi.org/10.1128/mbio.00010-20).

Libraries were sequenced at the UMass Medical School Deep Sequencing Core Facility on a HiSeq 4000. Raw data are available on GEO, accession number GSE244235. Reads were demultiplexed with Cutadapt (Martin, M. 2011 EMBnet.joural 17(1) 10-12) and aligned to the reference genome NC_000962 with BWA mem (Li,H. et al. 2010 Bioinformatics 26(5). Read count tables were generated with featureCounts (Liao, Y. et al. 2014 Bioinformatics 30(7) 923-930) and used for Principal Component Analysis and differential expression analysis with DESeq2 (Love, M. I. et al. 2014 Genome Biol. 15(12) 550). Each treatment was compared to the time-matched untreated control. Genes with adjusted p values<0.05 and log₂ fold changes>1 or <−1 were considered to be differentially expressed. Gene Set Enrichment Analysis was done with clusterProfiler (Wu, T. et al. 2021 Innovation (Camb) 2(3), 10014) using annotations obtained from AmiGO (Carbon, S. et al. 2009 Bioinformatics 25 92) 288-289) (https://amigo.geneontology.org/amigo).

### Extraction, Fractionation, and Isolation of Composition 1

A. afra leaf material 2.1 kg was ground and extracted in dichloromethane (DCM) (1 g:20 mL, VWR International, Radnor, PA, USA) under sonication for 30 minutes. The supernatant was decanted, and the process repeated two additional times. The total supernatant was collected and dried under reduced pressure, yielding 193.5 g crude extract. The crude extract was fractionated on a Biotage Selekt flash chromatography system with a Biotage Sfar HC Duo 350 g column (Biotage, Uppsala, Sweden) and using the ternary solvent system hexane (A), chloroform (B), and methanol (C) and the following (A:B:C) gradient at 200 mL/min: at 0 column volumes (OCV), 100:0:0; 3CV, 0:100:0; 8CV 0:100:0; 9CV 0:95:5; 14CV 0:95:5; 34CV 0:89:20. The flash separation yielded 5 pooled fractions, F1-F5. (Table 1).

TABLE I

| Extract, fraction, | MIC^{a} (mg/mL; Mtb strain |
|---|---|
| or compound | mc²6230, aerobic growth) |
| A. afra crude extract | 1.25-5^{b} |
| Fraction F1 | 1.25 |
| Fraction F2 | 0.3125 |
| Fraction F3 | 1.25 |
| Fraction F4 | >10 |
| Fraction F5 | >10 |
| Subfraction HAB1 | 1.25 |
| Subfraction HAB2 | 1.25 |
| Subfraction HAB3 | 0.625 |
| Subfraction HAB4 | 0.3125 |
| Subfraction HAB5 | 0.3125 |
| Subfraction HAB6 | 0.625 |
| Subfraction HAB7 | 0.3125 |
| Subfraction HAB8 | 0.625 |
| Subfraction HAB9 | 0.3125 |
| Subfraction HAB10 | 0.3125 |
| Subfraction HA14 | 0.625 |
| Subfraction HA16 | 0.625 |
| Subfraction HA17 | 2.5 |
| Subfraction HB13 | 1.25 |
| Subfraction HB14 | 2.5 |
| Subfraction HB15 | 5 |

Fraction F2 was subfractionated on a reverse phase flash column (Biotage Sfar C18 120 g) on the Selekt system with the ternary solvent system acetonitrile (A), water (B), and chloroform (C) and the following (A:B:C) gradient at 50 mL/min: at 0 column volumes (OCV), 2:98:0; 2 CV, 2:98:0; 5 CV, 5:95:0; 25CV, 85:15:0; 30 CV, 85:15:0; 31CV, 100:0:0; 41CV, 75:0:25; 42CV, 60:0:40; 47CV, 0:0:100, 49CV, 0:0:100. Fractionation method was performed twice and overlapping fractions combined, to yield 16 subfractions, HAB1-10, HA14, HA16, HA17, and HB13-15.

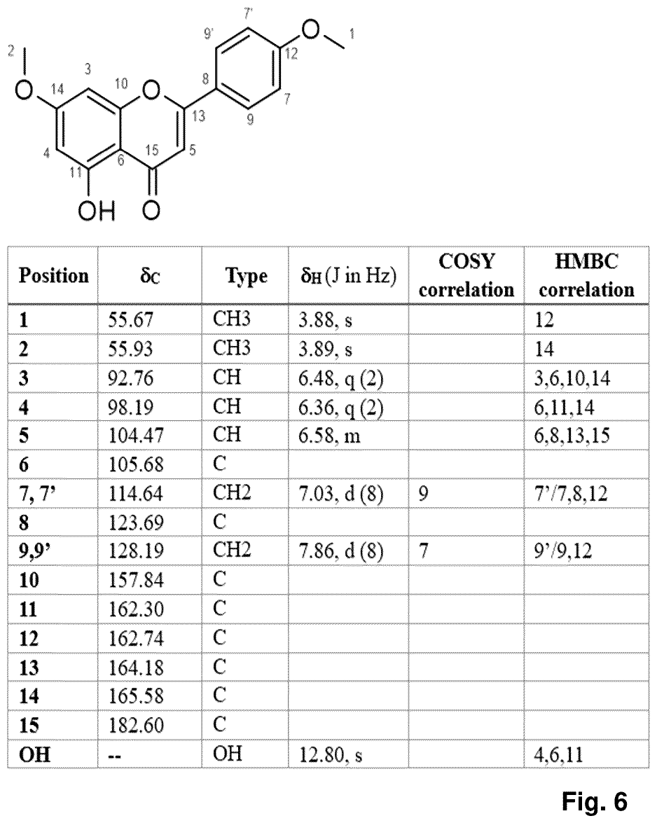

Composition 1 was determined to be 5-Hydroxy-4′,7-dimethoxyflavone (1): the purified dried material was a yellowish solid powder; ¹H and ¹³C NMR (FIG. 6); HRESIMS m/z 299.0914 [M+H]+(molecular weight calculated as C₁₇H₁₄O₅, 299.0919).

### Toxicity Testing in an Animal Model

Toxicity of extracts and fractions was measured using the whole organism brine shrimp assay, a method that is known from previous studies to show close correlation for plant extracts and rodent data (Lagarto Parra, A. et al. 2001 Phytomedicine 8 (5) 395-400). To hatch shrimp for testing, Aquatic Foods Great Salt Lake Brine Shrimp Eggs (50 mg) were added to 100 mL of 4% w/v Instant Ocean (Spectrum Brands Pet LLC, AQ-SS1510/1642606) and 3 mg baker's yeast in a 150 mL beaker, covered in aluminum foil, incubated for 48 hr at 28° C. with continuous fluorescent light, and sparged with ambient air at 0.5 LPM. Into individual 3 mL glass vials 10 shrimp were added using a 9 in glass Pasteur pipette and volume brought to 500 μL with Instant Ocean. To each vial a 10 mL aliquot of a 1:2 serial dilution of HAB9 (0-2 mg/mL) diluted in DMSO was added with 490 mL of Instant Ocean to bring the final volume to 1 mL. For hot water and DCM extracts the test range was the extract equivalent of 0-8 mg/mL of dried leaves with dilutions made in water and DMSO, respectively. Hot water extracts were pH adjusted to 7.5 with NaOH. After 24 h incubation, average % viability was calculated by counting the live shrimp per vial. There were three replicates per concentration, and the analysis was repeated three times.

### Mass Spectrometry and Metabolomics Analysis

Ultra-high Pressure (UP) LC-MS data were acquired using an Orbitrap Fusion Lumos Tribrid mass spectrometer (Thermo Scientific, Waltham, MA, USA) with an electrospray ionization source coupled to a Vanquish UHPLC system (Thermo Scientific). Injections (5 μL) were separated by reverse-phase UPLC using an Acquity BEH C18 column (150×2.1 mm, 1.7 μm particle size (Waters Corporation, Milford, MA, USA) held at 55° C. with a flow rate of 100 μL/min. The following binary solvent gradient was employed with solvent A (LC-MS grade water with 0.1% formic acid), and solvent B (LC-MS grade acetonitrile): initial isocratic composition of 97:3 (A:B) for 1.0 min, increasing linearly to 85:15 over 4 min, increasing linearly to 5:95 over 11 min, followed by an isocratic hold at 5:95 for 2 min, gradient returned to starting conditions over 0.1 min and held for 1.9 min. The positive ionization mode was utilized over a full scan of m/z 100-1000 with the following settings: spray voltage, 3.5 kV; IT tube temperature, 275° C.; vaporizer temperature, 75° C.; sheath gas flow and auxiliary gas flow, 25 and 5 units, respectively.

### Nmr Analysis

¹H and ¹³C NMR spectra were acquired with a Bruker AVIII-500 and Bruker NEO 600 (500 and 600 MHz, respectively; Bruker Corporation, Billerica, MA, USA) equipped with a triple resonance TCI single axis gradient cryoprobe running TopSpin 3.2 operating software. NMR chemical shift values were referenced to residual solvent signals for CDCl₃ (δ_{H} 7.26 ppm). To collect NMR data, samples were resuspended in CDCl₃ (Cambridge Isotope Laboratories, Andover, MA, USA), and the following experimental data were collected: ¹H, ¹³C, ¹H-¹H COSY, DEPT₁₃₅, HSQC, and HMBC. The NMR data for 1 have been deposited in the Natural Products Magnetic Resonance Database (np-mrd.org; Wishart, D. S. et al. 2022 Nucleic Acids Res. 50 (D1) D665-D677).

### Biochemometric Analysis

The peak area of extracted ions was used as the variable for chemometrics analysis. SIMCA 17.0 software (Umetrics, Umea, Sweden) was used for chemometrics analysis. Prior to chemometrics analysis, the data were range scaled. No data transformation was performed. Orthogonal partial least squares-discriminate analysis (OPLS-DA) was applied for multivariate modeling. Models were evaluated using the score plot, R²X, R²Y, and Q² metrics. Cross-validation using the leave-one-out technique was used. An S-line plot was used to identify the important variables responsible for predicting anti-tuberculosis activity in the OPLS-DA model.

### Statistical Analysis of Toxicology and RNAseg Data

For the brine shrimp lethality test, an average of three independent replicate experiments each with three technical replicates per concentration were used to calculate an average IC₅₀ using GraphPad Prism version 10.0.0. RNAseq statistical analysis is described in that section.

It is shown from the data in examples herein that Artemisia extracts can kill Mtb for several logs of killing and over a period of days. The killing observed was regardless of the carbon source used for Mtb growth, and, most important, to a greatest extent when Mtb is hypoxically grown. Mtb metabolizes a variety of different sources of carbon (de Carvalho, L. P. S. et al., 2010 Chem. Biol. 17(10) 1122-1131; Gould, T. A. et al., 2006 Mol. Microbio. 61(4) 940-947; Koh et al., 2022, Ibid.; Marrero, J. et al., 2010 Proc. Natl Acad. Scie 107(21) 9819-9824; Munoz-Elias, E. J. et al., 2005 Nat. Med. 11(6) 638-644; Zimmermann, M. et al., 2017 mSystems 2(4), e00057-00017). The results in examples herein showed that although growth rates and yields were the same with each of carbon sources dextrose, glycerol, and cholesterol, addition of the extracts altered the response by showing not growth but decreased survival by several logs of cell death. Furthermore, results were different between A. annua and A. afra-treated Mtb cultures, suggesting that there may be two different mechanisms of killing action in different carbon sources in response to each Artemisia sp.

Plants have a long history of providing bioactive compounds for vital and novel therapeutics. Inhibitory effects have been reported for A. annua and A. afra extracts, having activity against growth of Mtb (Cantrell et al., 1998, Ibid.; Martini et al., 2020, Ibid.; Mativandlela et al., 2008. Ibid.; Ntutela et al., 2009. Ibid.; Uba et al., 2003, Ibid.). Most prior studies were in vitro, however, Ntutela et al. also included rodent testing. Although those in vitro tests showed a DCM extract MIC of 290 μg/mL against Mtb H37Rv, and a subfraction activity (fraction C8) that at 2 μg/mL was nearly 100 times more potent than the original extract, there was no efficacy of either the DCM or hot water extract in vivo in Mtb-infected mice.

Ntutela et al. studies showed considerable variation among their extraction preparation methods and that may be a reason for loss of activity in vivo. As example, 200 g of dried leaves were boiled in 4L water for 30 min, filtered and then freeze dried to yield a hot water infusion. The traditional hot water extraction is boiled for 5-10 min at 5 g/L or steeped for 5-10 min in the boiled water. In our unpublished studies increasing the dry mass of Artemisia:water ratio beyond 10 g/L results in >50% loss of extractables (unpublished). Furthermore, increasing the leaf g/L beyond˜10 g/L causes a significant decline in extractables (van der Kooy, F. et al., 2011 Planta Med 77(15) 1754-1756), e.g., ART recovery declined from 62 to 29% when leaf:hot water ratios increased from 20 to 50 g/L. Ntutela et al. used a water extract of 50 g/L with 30 min boiling and lyophilized to dryness. In our experience with A. annua, ART was not fully recovered when our lyophilized tea infusion was reconstituted in water resulting in about a 50% loss, so for experiments we never use reconstituted lyophilized tea. When compared to our tea MIC of ˜1.5 mg/mL the Ntutela et al. water extract had neither in vitro nor in vivo activity (Table 1), results consistent with a possible loss of activity of the water extract in vivo. A similar argument can be made for the DCM extract of Ntutela et al. because they extracted at 2 g/10 mL then twice more but each at half the original solvent volume (Table 1).

In contrast, data obtained in examples herein use material that was extracted 1 g/20 mL DCM for 30 min in a sonicating water bath at room temperature and extracted twice again using the same volumes (Martini et al., 2020). The data herein for A. afra DCM extract had a MIC of 2.5 mg/mL (MAL), more than a 100-fold greater potency than the 290 mg/mL DCM MIC reported by Ntutela et al. (Ntutela et al., 2009, Ibid.). One might question if the extracts even reached the lungs of the treated mice, however, an ADME (absorption, distribution, metabolism, excretion) study of rats treated per os with A. annua showed considerable amount of the marker drug ART reached the lungs (Desrosiers et al., 2020, Ibid.). Ntutela et al. did not gavage their animals with the Artemisia extracts, but instead mixed them into the feed for ad libidum consumption, which results in compromised dosing.

Mtb carbon metabolism can be a target for the development of therapeutics due to the dependency of mycobacterial growth, dormancy, and virulence on metabolic pathway differences between Mtb and humans. Moreover, the potency of several drugs is influenced by carbon metabolism. In this sense, Kalia and collaborators reported that glycerol supplementation interfered with the potency of drugs targeting cytochrome bc₁:aa₃ in mycobacteria (Kalia et al., 2019, Ibid.).

Additionally to Mtb and tuberculosis, many other mycobacterial species cause disease conditions in humans and other animals, exemplary species including without limitation, M. abscessus, M. avium-intracellulare, M. chelonae, M. fortuitum, M. kansasii, M. xenopi, M. marinum, M. scrofulaceum, and M. szulgai. Such species infect many sites within the body but primarily cause pulmonary disease, cervical lymphadenopathy, and localized skin and soft tissue lesions.

Data from examples herein show major growth differences of Mtb in different carbon sources when exposed to A. annua and A. afra extracts. Cholesterol plays a pivotal role in the infectivity and virulence of Mtb (Abuhammad, A. 2017, Br. J. Pharmacol. 174(14) 2194-2208). Mtb uses cholesterol as its main carbon source and any other compounds or carbon sources that hinder cholesterol metabolism can inhibit Mtb growth resulting in carbon starvation and metabolic intoxication subsequently causing an imbalance in central metabolism. Chang and Guan confirmed that cholesterol and fatty acid are the main carbon sources that Mtb uses during infection (Chang, D. P. S. et al., 2021 Metabolites 11(2) 88).

Three main putative operons affect cholesterol entry and catabolism in mycobacteria: mce4, igr, and the hsaACDB cluster (Abuhammad, 2017 Ibid.). The mammalian cell entry mce4 operon is involved in cholesterol uptake and use in Mtb and deletion or disturbance of the mce4 operon results in a growth defect. The intracellular growth igr operon is essential for growth and virulence in macrophages and is involved in cholesterol metabolism and deletion of this operon results in growth defects due to toxic metabolite accumulation. The hsaACDB cluster is involved in the degradation of the cholesterol sterol-ring and is essential for the intracellular survival of mycobacteria within the macrophage. Any change in carbon source other than cholesterol, therefore, disrupts the cholesterol degradation pathway resulting in decreased Mtb growth. Furthermore, Noy et al. (Noy, T. et al., 2016 J. Biol. Chem. 291(13) 7060-7069) studied the effect of different carbon sources other than glucose on the activity of glucose-6-phosphate, which is the main driver of glycolysis and observed that the activity of that enzyme decreased when Mtb was grown in carbon sources other than glucose. For example, glucose-6-phosphate levels were 5-fold higher in cells grown on glucose compared to acetate. As A. annua and A. afra extracts contain different compounds they may hinder Mtb's central metabolic thereby reducing growth in the presence of the extracts. Future studies could involve tracking labeled metabolites to measure changes in Mtb metabolism in different carbon sources in the presence of Artemisia extracts.

Hypoxia and the gradual depletion of oxygen is a key element in granuloma development by Mtb in humans and an important consideration in the design of therapeutics useful for treating tuberculosis (Gibson, S. E. et al., 2018 Pathogens 7(4) 88). Several in vitro models to obtain non-replicating M. tuberculosis have been developed and are based on reducing oxygen availability and nutrient starvation. One of the most used methods is the Wayne model, in which non-replicating Mtb is obtained by gradually adapting stirred aerobic cultures to hypoxia through a self-generated oxygen depletion gradient (Sohaskey, C. D. et al. 2015 Mycobacteria Protocols, Springer 201-213).

Several lines of evidence link tuberculosis and inhibition of Mtb growth/metabolism with hypoxic conditions within the host. Tuberculosis infections are preferentially associated with the most oxygen-rich sites in the body (Adler-Shohet, F. C. et al., 2014 Pediat. Inf. disease J. 33(6) 664-666). Reduced levels of O₂ may limit Mtb growth in vivo. This may explain why recrudescent tuberculosis occurs most often in the upper lobes of the lung, the single most-oxygenated region of the body. Lim et al. (Lim, J. et al., 2021 Proc. Natl. Acad. Sci 118(35) e2105800118) show that phosphoenolpyruvate (PEP) is almost completely depleted in Mtb under hypoxic conditions. A loss of PEP reduces PEP-carbon flux toward multiple pathways essential for the replication of Mtb. Metabolomics profile of Mtb collected under hypoxia showed accumulation of intermediates in glycolysis and the reductive branch of the TCA cycle, with reciprocal depletion of PEP and oxidative branch intermediates of the TCA cycle such as α-ketoglutarate. Under hypoxia PEP depletion may affect multiple cellular metabolic processes that are involved in Mtb metabolic remodeling (Marrero et al., 2010, Ibid.).

Data from examples herein show that the killing curves for growth of Mtb under hypoxia was substantially reduced by several logs of killing in presence of A. annua and A. afra extracts. Thus, if Mtb is exposed to different environmental stresses e.g., hypoxic conditions along with Artemisia extracts it is possible that its growth can be controlled to some extent. Under hypoxic conditions and especially in the presence of plant extracts there might be changes in the Mtb metabolic pathways, especially the TCA cycle. Any change in the TCA cycle will likely lead to a change in gene expression in the electron transport chain, which may cause a reduction in Mtb growth under our tested conditions. The activity of various anti-tubercular drugs is affected under such stressful conditions. For example, isoniazid (INH) is one among the four first-line drugs used in the treatment of tuberculosis and in vitro kills actively growing Mtb vegetative bacilli, but possesses little or no activity against Mtb under conditions of nutrient starvation or progressive oxygen depletion (Raghunandanan,S. et al., 2018 J. antibiotics 71(11) 939-949).

Examples herein show that Mtb growth was significantly reduced in the presence of A. annua and A. afra extracts when grown in glycerol, dextrose or cholesterol as sole carbon sources and several logs of killing were observed in Mtb grown under hypoxic conditions.

It was hypothesized that there are other phytochemicals in A. afra that are not artemisinin and that are responsible for the bioactivity. As shown in examples herein, A. afra activity could be shown in Mtb grown in vitro with each of several different carbon sources and grown under hypoxia, a condition frequently encountered by Mtb during human disease (Kiani, B. H. et al. 2023 Pathogens 12(2) 227, incorporated herein in its entirety by reference). These results herein led to a search for A. afra compounds with particular clinical potential, as many existing drugs are carbon-source-dependent and/or have poor activity against hypoxia-arrested Mtb.

Configurations herein performed transcriptomic profiling to assess the biological impact of Artemisia extracts on Mtb, then investigated the phytochemical content of A. afra responsible for its activity. Examples herein used the methods of biochemometry, fusing untargeted metabolomic profiling with in vitro bioassay data to identify potential antimycobacterial compounds. A DCM extract of A. afra was fractionated and assayed to determine minimum inhibitory concentration (MIC) as well as bactericidal activity against Mtb. The active fraction (HAB9) was separated chromatographically, and the metabolomic profile and anti-Mtb assay data were combined in a supervised multivariate model, to highlight the presence of a methoxylated flavone structure 1. The active fraction, containing>97% Compound 1, was shown to possess bactericidal activity against Mtb both during aerobic growth and in arrest of hypoxia-induced growth.

### The Transcriptomic Impact of Artemisia Extracts and Artemisinin

Upon exposure to drugs, bacteria generally up- and down-regulate sets of genes as they attempt to cope with the specific stressors imposed by the treatment. Transcriptomic profiling can therefore provide insight into the specific effects of different drugs. To assess and compare the transcriptomic responses of Mtb to Artemisia extracts, we performed RNAseq on cultures exposed to DCM extracts of A. annua and A. afra, as well as to pure artemisinin. Each of the three treatments was applied at three levels: a lethal dose, a sub-lethal dose that inhibited most growth, and a dose that slowed growth but did not stop it. The concentrations of drug and extract that produced these phenotypes were determined by plating for CFU. The transcriptomic impacts of the lethal doses were then assessed after four hours, to prevent confounding by the accumulation of dead cells, while the impacts of inhibitory and sub-inhibitory doses were assessed after 24 hours.

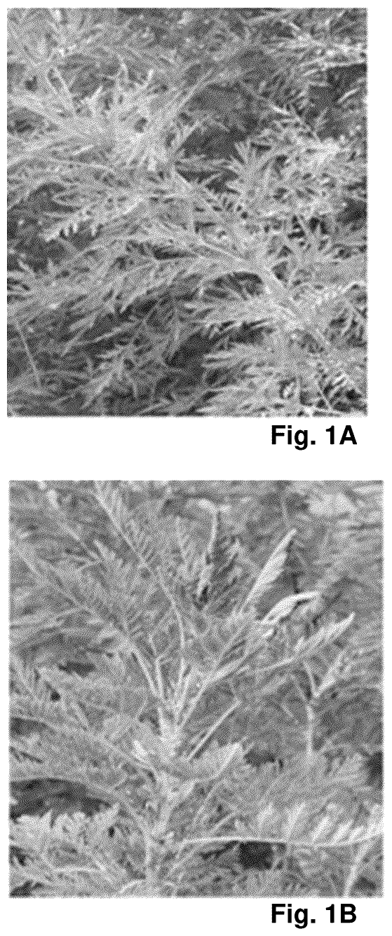

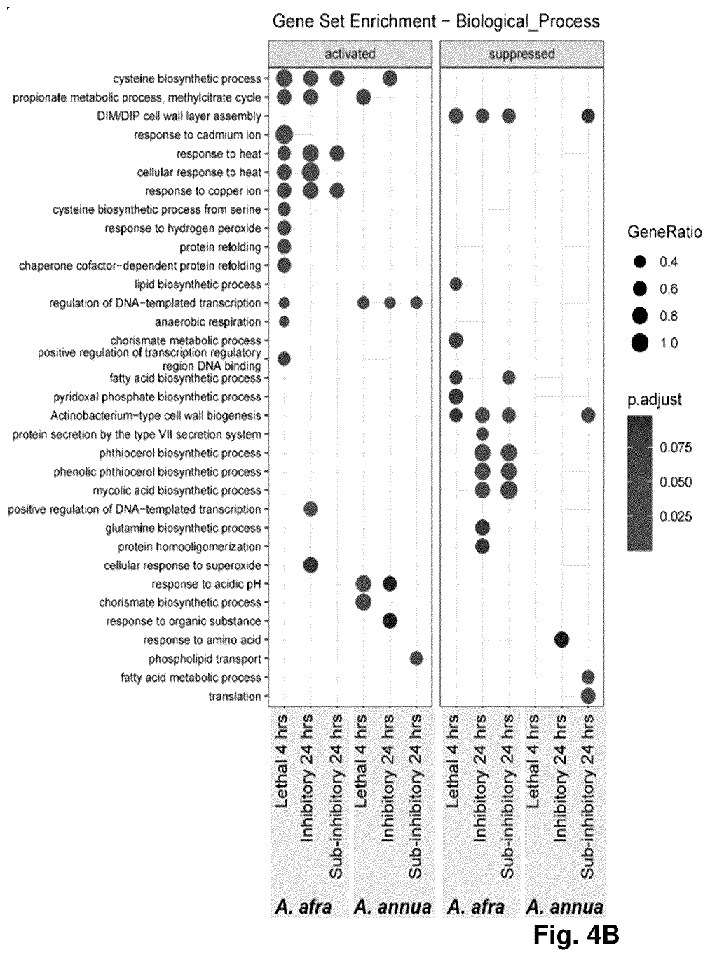

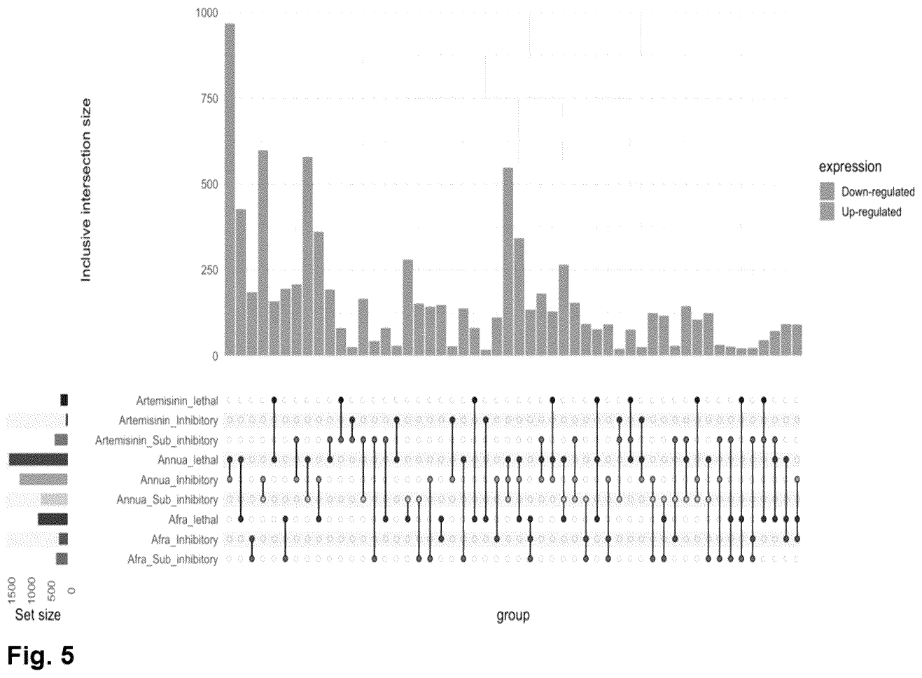

The overall transcriptomic profiles clustered by type of treatment (A. annua, A. afra, artemisinin) rather than by the severity of the treatment (FIG. 1A). This indicated that the profiles largely reflected treatment-specific responses rather than non-specific responses such as slowed growth that are shared between treatments. The transcriptomic impacts of the three treatments were distinct from one another (FIG. 4A), indicating that A. annua and A. afra affect Mtb through distinct mechanisms, and that A. annua has substantial impacts on Mtb through artemisinin-independent mechanisms. The overall transcriptomic impact of artemisinin was smaller than that of the plant extracts, as revealed by clustering (FIG. 4A) and by the number of differentially expressed genes compared to no-drug controls (FIG. 5). This may reflect artemisinin being a single compound that likely has a single mechanism of action towards aerobically growing cells, while the extracts likely contain multiple compounds with different mechanisms of action.

To investigate the specific physiological impacts of each treatment, we performed Gene Set Enrichment Analysis (Wu, T. et al. 2021 Ibid.) to identify pathways that were disproportionately affected. The enriched pathways were largely distinct for A. annua and A. afra extract treatments, consistent with the idea that their primary mechanisms of action against Mtb are distinct (FIG. 4B). No pathways were disproportionately affected by artemisinin. Among the pathways induced by A. afra treatment, several were consistent with responses to oxidative stress (e.g., cysteine biosynthesis, response to cadmium, response to heat, response to copper, response to hydrogen peroxide, and protein refolding). Notably, many existing antibiotics are thought to induce oxidative stress indirectly, suggesting potential for synergy with A. afra. The pathways that were downregulated in response to A. afra treatment included several involved in cell envelope biosynthesis. The pathways up- and down-regulated in response to A. annua treatment were less easily categorized.

### Fractionation and Activity Testing of an A. afra Extract

Given the clearly artemisinin-independent activity of A. afra, we sought to identify its active compounds. Flash chromatography was used to create five fractions. One of these, F2, had the lowest MIC against Mtb strain mc²6230 (H37Rv ARD1 ApanCD, Sambandamurthy V. K. et al. 2006 Ibid.) (Table I) and also had the most potent bactericidal activity. However, the fractions were too complex to adequately model their bioactive chemistry via a PLS or OPLS-DA model. Botanicals have been known to possess too many constituents per fraction after a preliminary round of fractionation, so subsequent subfractionation must be undertaken to simplify the chemistry of each sample thereby facilitating a more robust biochemometric model (Britton, E. R. 2018, J. Nat. Products 81(3) 484-493). Thus, the most active fraction, F2, was selected for further subfractionation on a reverse phase flash system, yielding 16 subfractions (HAB1-10, HA14, 16, 17, and HB13-15). Of these, subfraction HAB9 had among the lowest MICs (Table 1) and strong bactericidal activity against aerobically growing Mtb. The activity of HAB9 against Mtb in hypoxia-induced growth arrest was so potent that no colonies were observed after two days of treatment, which was the first time-point assessed after addition of the fraction.

### Biochemometric Identification of Anti-TB Compound 1

Biochemometric modeling of the F2 subfractions was achieved using their untargeted metabolomics profiles of and their classification as ‘active’ or ‘inactive’ based upon the normoxic MIC data. An orthogonal partial least squares-discriminate analysis (OPLS-DA) supervised model led to the correlation and covariance of the features (ions) with the bioactivity categorization. The model gave a good level of fitness (R2X=0.828, R2Y=0.778) and good predictivity (Q2=0.798), and the resulting S-line plot yielded several metabolites of interest (FIG. 3A). The S-line plot highlights variables with a high value of correlation (shown by the red coloration), and covariance (value along the y-axis), play a crucial role in the OPLS-DA discriminatory model between active and inactive. The main covarying feature had an m/z of 299.0841, which was observed as the main compound in the subfraction HAB9 and was isolated as 1 (FIG. 6). Compound 1 comprised 97.4% of HAB9 by mass, and therefore the activity observed in FIGS. 3A-3D is attributed to Compound 1.

Compound 1 had a molecular formula of C₁₇H₁₄O₅, based on the protonated HRESIMS ion with an m/z of 299.0914. Inspection of the ¹H, ¹³C, heteronuclear single quantum correlation (HSQC), and heteronuclear multiple bond correlation (HMBC) NMR data permitted the identification of 1 as 5-hydroxy-7-methoxy-2-(4-methoxyphenyl)chromen-4-one (e.g., 5-hydroxy-4′,7-dimethoxyflavone); FIG. 6. This compound was one of the more prevalent flavonoids in the A. afra DCM extract, with a concentration of 2.29 mg/g dried leaf. This flavone was previously demonstrated in other Artemisia species (reviewed in Valant-Vetschera, K. M. et al. 2003 Biochem. System. And Ecol. 31(5) 487-498), but this is the first reporting of this compound from A. afra, and its first reported bioactivity.

### Cytotoxicity Studies

A. afra is not considered a toxic medicinal plant (Kane, N. F. et al., 2019 Adv. Biosci. Bioeng. 7(4) 64), and further, the toxicity of HAB9 (1), and other extracts of the plant was evaluated using the whole animal brine shrimp assay (Table 2). The IC₅₀ values of the traditional hot water infusion (tea), a DCM extract, and HAB9 were 4.27, >2.5, and 0.023 mg/mL, respectively. The HAB9 fraction, which is identified as 5-hydroxy-4′,7-dimethoxyflavone (1), appears to be more toxic in brine shrimp than in mammalian cell cultures where it was deemed nontoxic up to the maximum tested concentration of 0.029 mg/mL. Further toxicity testing in additional animal systems is envisioned for compound 1 and related fractions.

A. afra extracts showed potent activity against Mtb in vitro and were observed to be acting along distinct pathways compared to either A. annua or artemisinin. Subsequent biochemometric analysis identified the methoxylated flavone 1 as one of the plant's phytochemicals active against Mtb. Exposure to 1 substantially reduced viable cultures of Mtb in both replicating and non-replicating stages, which is significant given the insensitivity of non-replicating Mtb to most drugs. While potential toxicity may limit use of 1 as a direct anti-Mtb compound, 1 may serve a basis for the design of other novel antimycobacterial drugs. The position of the methoxy groups suggest potential future structure-activity studies to determine optimal positioning and derivatization of flavones and structure modification to improve efficacy and reduce toxicity.

Furthermore, other fractions contain additional A. afra phytochemicals that are also antimycobacterial. The data herein show that A. afra is a source of effective botanical compounds, and potential structural designs of novel agents, for use in the prevention and treatment of tuberculosis.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method of use of 5-hydroxy-7-methoxy-2-(4-methoxyphenyl)chromen-4-one (e.g., 5-hydroxy-4′,7-dimethoxyflavone), identified herein as composition 1, for inhibiting growth and viability of bacteria, the method comprising contacting cells of the bacteria with composition 1 and observing growth and viability in comparison to control bacteria not contacted.

2. The method according to claim 1, wherein the bacteria are cells of genus Mycobacterium.

3. The method according to claim 2, wherein the cells are M. tuberculosis.

4. The method according to claim 2, wherein the cells are at least one selected from the group of M. abscessus, M. avium-intracellulare, M. chelonae, M. fortuitum, M. kansasii, M. xenopi, M. rarinur, M. scrofulaceum, and M. szulqai.

5. The method according to claim 1, wherein contacting the bacteria comprises treating an infection in a mammalian subject.

6. The method according to claim 5, wherein the subject is a human patient.

7. The method according to claim 1, wherein prior to the step of contacting, the composition 1 is formulated with at least one additional component selected from the group of: a second antibacterial agent, a pharmaceutically acceptable buffer, an emollient, and a carrier.

8. A pharmaceutical composition having anti-mycobacterial activity comprising composition 1 formulated with a pharmaceutically acceptable carrier, in an effective dose.

9. The pharmaceutical composition according to claim 8, for administration to a mammalian subject by a route selected from the group of: intranasal, oral, sublingual, intravenous, intradermal, transdermal, topical, intra-muscular, and subcutaneous.

10. The pharmaceutical composition according to claim 8, wherein the anti-mycobacterial activity for cells grown in limiting oxygen is enhanced compared to aerobically grown cells.

11. The pharmaceutical composition according to claim 8, having a minimal inhibitory concentration (MIC) for M. tuberculosis of less than 300 micrograms/ml; less than 100 micrograms/ml; less than 50 micrograms/ml.

12. A method of making 5-hydroxy-7-methoxy-2-(4-methoxyphenyl)chromen-4-one (e.g., 5-hydroxy-4′,7-dimethoxyflavone) or composition 1 from leaves of an Artemisia plant species, the method comprising:
providing a sample of leaves of A. afra or A. annua;
grinding under sonication of the leaves under dichoromethane (DCM), thereby extracting the composition 1 into the supernatant;
fractionating the supernatant by flash chromatography and eluting with a gradient of solvents; and,
sub-fractionating the activity containing fraction by reverse phase flash chromatography, to obtain the composition 1.

13. The method according to claim 12, wherein providing the leaves is A. afra.

14. The method according to claim 12, wherein the sample is at least about 5 mg, about 5 grams, about 50 grams, about 500 grams, about 1 kg, about 2 kg.

15. The method according to claim 12, wherein eluting with a gradient of solvents is using mixtures of hexane, chloroform and methanol in a series of ratios.

16. The method according to claim 12, wherein reverse phase flash chromatography comprises eluting with mixtures of acetonitrile, water and chloroform in a series of ratios.

17. The method according to claim 12, wherein resulting composition 1 is a 5-hydroxy-4′,7-dimethoxyflavone in a yellowish powder.

18. The method according to claim 17, further comprising analyzing purity and identifying chemical structure.

19. The method according to claim 18, wherein identifying yields the structure of 5-hydroxy-7-methoxy-2-(4-methoxyphenyl)chromen-4-one.

20. The method according to claim 18, wherein analyzing the purity of the yellowish powder yields at least 90%, at least 95%, at least 97%.

21. The method according to 20, further comprising diluting a sub-fraction having activity or dissolving the yellowish powder and diluting for performing toxicity studies.

22. The method according to claim 21, wherein performing toxicity studies comprises at least one selected from the group of: contacting cells of mammalian origin in vitro; contacting mammalian subjects in vivo; and contacting non-mammalian subjects in vivo.
